(19) United States
(12) Patent Application Publication
(10) Pub. No.: US 2007/0167965 A1
(43) Pub. Date: Jul. 19, 2007

(54) Ultrasonic medical instrument

(75) Inventor: Kevin L. Houser, Springboro, OH (US)

Correspondence Address: Mark P. Levy, 2000 Courthouse Plaza, NE, 10 West Second Street, Dayton, OH, 45402-1758, US

(73) Assignee: Ethicon Endo-Surgery, Inc.

(21) Appl. No.: 11/326,256

(22) Filed: Jan. 5, 2006

(51) Int. Cl. A61B 17/32

(52) U.S. Cl. 606/169

(57) ABSTRACT

A first ultrasonic medical instrument includes an ultrasound transducer, a medical ultrasonic blade, and a tube adapted to contain a liquid. A first tube end is adapted to have the liquid proximate the first tube end, when the tube contains the liquid, be ultrasonically vibrated to generate an ultrasonically-vibrating energy wave. The ultrasonic blade is acoustically connected to the liquid proximate a second tube end, when the tube contains the liquid. A second ultrasonic medical instrument includes a heat pulse initiator, a medical ultrasonic blade, and a tube adapted to contain a liquid. The heat pulse initiator is adapted to output heat pulses having an ultrasonic pulse frequency. A first tube end is adapted to receive the heat pulses outputted by the heat pulse initiator, wherein the heat pulses have an energy sufficient to create an ultrasonically-vibrating energy wave in the liquid, when the tube contains the liquid. The ultrasonic blade is acoustically connected to the liquid proximate the second tube end, when the tube contains the liquid.

### FIELD OF THE INVENTION

The present invention is related generally to medical instruments, and more particularly to an ultrasonic medical instrument having a medical ultrasonic blade.

### BACKGROUND OF THE INVENTION

Known ultrasound medical instruments include those having an ultrasonic surgical blade in the form of a titanium rod. The ultrasonic surgical blade is used for cutting patient tissue and for sealing blood vessels to stop hemorrhaging. The ultrasonic surgical blade is attached to a handpiece and acoustically connected to an ultrasound transducer contained in the handpiece. The ultrasound transducer is operatively connected to an ultrasound generator by a cable. Articulating medical catheters, such as endoscope tubes, are also known.

Still, scientists and engineers continue to seek improved ultrasonic medical instruments having an ultrasonic medical blade.

### SUMMARY OF THE INVENTION

A first expression of a first embodiment of the invention is for an ultrasonic medical instrument including an ultrasound transducer, a medical ultrasonic blade, and a tube. The tube is adapted to contain a liquid and has a first tube end and a second tube end. The first tube end is adapted to have the liquid proximate the first tube end, when the tube contains the liquid, be ultrasonically vibrated to generate an ultrasonically-vibrating energy wave. The ultrasonic blade is acoustically connected to the liquid proximate the second tube end, when the tube contains the liquid.

A first expression of a second embodiment of the invention is for an ultrasonic medical instrument including a heat pulse initiator, a medical ultrasonic blade, and a tube. The heat pulse initiator is adapted to output heat pulses having an ultrasonic pulse frequency. The tube is adapted to contain a liquid and has a first tube end and a second tube end. The first tube end is adapted to receive the heat pulses outputted by the heat pulse initiator. The ultrasonic blade is acoustically connected to the liquid proximate the second tube end, when the tube contains the liquid.

Several benefits and advantages are obtained from one or more of the embodiments of the invention. In one example of the first and/or the second embodiment, the tube is a flexible tube and is controllably bent by a user during a medical procedure to allow the medical ultrasonic blade to more easily access a target site in a patient.

The present invention has, without limitation, application with straight or curved ultrasonic surgical blades, with or without clamping arms, and further in hand-activated instruments as well as in robotic-assisted instruments.

### BRIEF DESCRIPTION OF THE FIGURES

FIG. 1 is a schematic view, with portions shown in cross section, of a first embodiment of an ultrasonic medical instrument of the invention, wherein the generator is located outside the housing containing the ultrasound transducer and wherein the sheath is a short sheath;

FIG. 2 is a view, as in FIG. 1, but of an alternate embodiment of the instrument of FIG. 1, wherein the generator is located inside the housing containing the ultrasound transducer and wherein the sheath is a long sheath;

FIG. 3 is a view, as in FIG. 1, but of an alternate embodiment of the instrument of FIG. 1, wherein the tube has a shoulder and wherein no sheath is present;

FIG. 4 is a schematic view, with portions shown in cross section, of a second embodiment of an ultrasonic medical instrument of the invention which includes a heat pulse initiator in the form of an end of a resistive or radio-frequency flexible wire; and

FIG. 5 is schematic view of an alternate embodiment of the heat pulse initiator and associated components of FIG. 4, wherein the heat pulse initiator is in the form of an end of a flexible laser fiber.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, examples, etc. can be combined with any one or more of the other following-described embodiments, examples, etc.

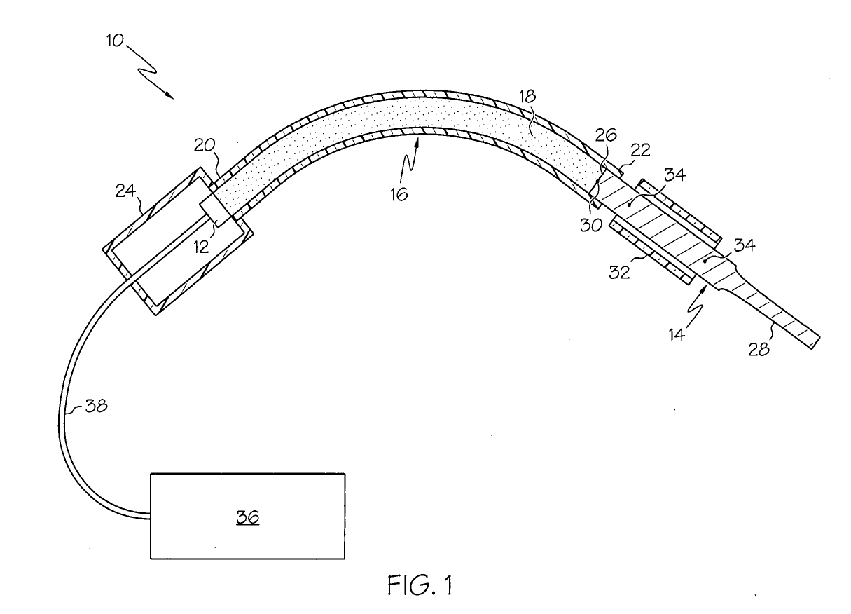

Referring now to the Figures, in which like numerals indicate like elements, FIG. 1 illustrates a first embodiment of the invention. A first expression of the embodiment of FIG. 1 is for an ultrasonic medical instrument 10 including an ultrasound transducer 12, a medical ultrasonic blade 14 (such as, without limitation, a titanium ultrasonic blade), and a tube 16. The tube 16 is adapted to contain a liquid 18 and has a first tube end 20 and a second tube end 22. The first tube end 22 is adapted to have the liquid 18 proximate the first tube end 20, when the tube 16 contains the liquid 18, be ultrasonically vibrated to generate an ultrasonically-vibrating energy wave. The ultrasonic blade 14 is acoustically connected to the liquid 18 proximate the second tube end 22, when the tube 16 contains the liquid 18. It is noted that the term “proximate” includes, without limitation, the word “at”. In one example, not shown, the tube is a rigid tube.

In one enablement of the first expression of the embodiment of FIG. 1, the tube 16 is a flexible tube and is filled with the liquid 18. In the same or a different enablement, the liquid 18 is an at-least-partially de-gassed liquid or an essentially de-gassed liquid. In one example, the de-gassed liquid is less prone to cavitation when ultrasonically vibrated than if not de-gassed. In the same or a different enablement, the liquid 18 is a pressurized liquid. In one example, the pressurized liquid is less prone to cavitation when ultrasonically vibrated than if not pressurized. In the same or a different enablement, the liquid 18 consists essentially of water or mineral oil.

In one construction of the first expression of the embodiment of FIG. 1, the ultrasonic blade 14 has a proximal blade end 26 and a distal blade portion 28, and the distal blade portion 28 is adapted to contact and medically treat patient tissue. In one variation, the ultrasonically-vibrating energy wave has a vibration antinode 30, and the proximal blade end 26 is disposed proximate the vibration antinode 30. In one illustration, the ultrasonically-vibrating energy wave is transmitted as pressure pulses through the liquid 18, and the ultrasonic blade 14 is held to the tube 16 in a manner which allows, when the pressure pulse is transmitted, the proximal blade end 26 to move and return the energy to motion.

In one modification, the ultrasonic medical instrument 10 includes a housing 24 containing the ultrasound transducer 12, wherein the tube 16 is attached to the housing 24. In one deployment, the housing 24 and the tube 16 essentially do not vibrate. In one example, the housing 24 and the tube 16 are adapted to be held by a user when the distal blade portion 28 contacts and medically treats patient tissue. In one arrangement, the ultrasonic medical instrument 10 includes an ultrasound generator 36 disposed outside the housing 24 and operatively connected to the ultrasound transducer 12 (such as by a long cable 38).

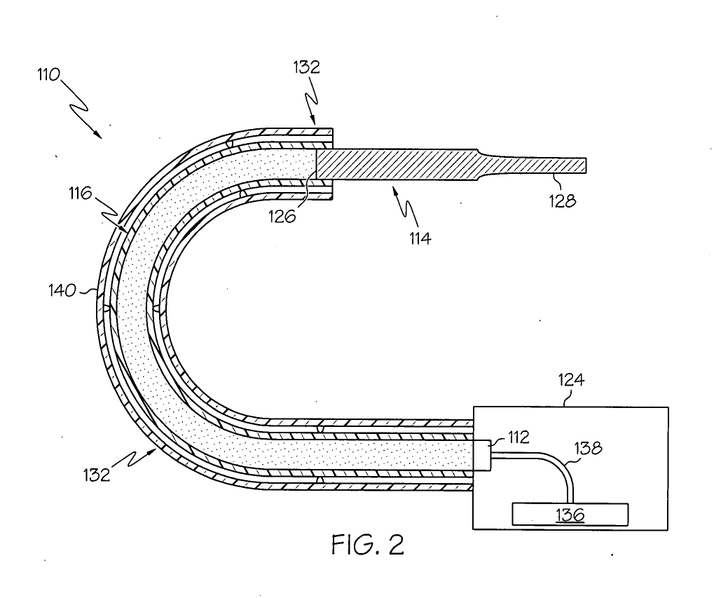

In a different example, as shown in the alternate embodiment of FIG. 2 and with the sheath 132 being omitted, the tube 116, but not the housing 124, is adapted to be held by a user when the distal blade portion 128 contacts and medically treats patient tissue. In a different arrangement, as shown in the alternate embodiment of FIG. 2, the ultrasonic medical instrument 110 also includes an ultrasound generator 136 disposed inside the housing 124 and operatively connected to the ultrasound transducer 112 (such as by a short cable 138).

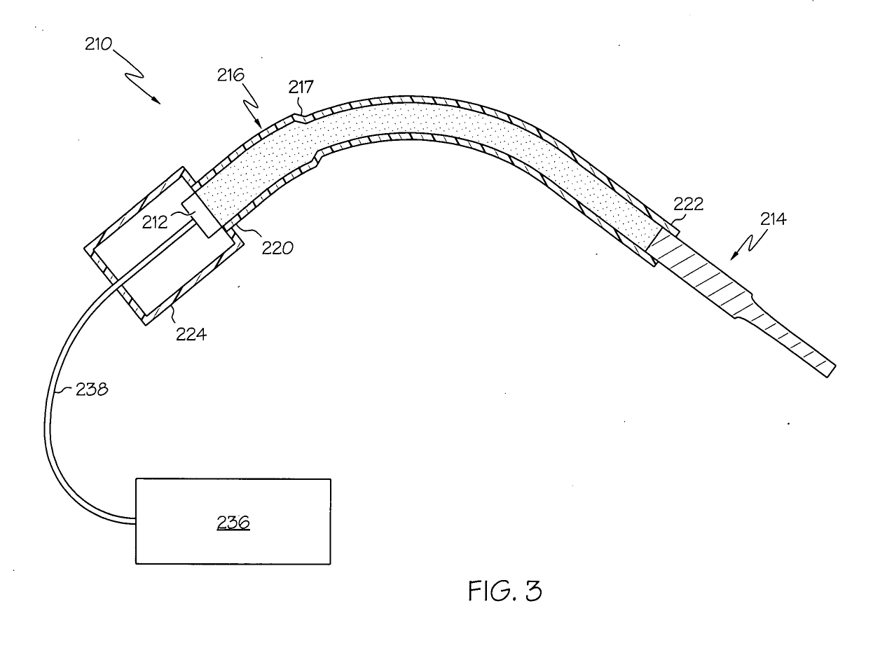

In one configuration, as shown in the alternate embodiment of FIG. 3, the tube 216 has a shoulder 217 between the first and second tube ends 220 and 222. In this configuration, the tube 216 has a first inside diameter between the first tube end 220 and the shoulder 217, and the tube 216 has a second inside diameter between the shoulder 217 and the second tube end 222, wherein the first diameter is not equal to the second diameter. This provides a gain step in the pressure pulse. The gain step is greater than unity when the first diameter is greater than the second diameter as shown in FIG. 3. The gain step is less than unity when the first diameter is less than the second diameter (not shown). The embodiment of FIG. 3 also shows the ultrasound transducer 212, the ultrasonic blade 214, the housing 224, the ultrasound generator 236, and the cable 238 of the ultrasonic medical instrument 210. It is noted that in the embodiment of FIG. 3, there is no sheath present.

In a first employment of the first expression of the embodiment of FIG. 1, the ultrasonic medical instrument 10 includes a sheath 32 surrounding and attached to the ultrasonic blade 14. In one example, the attached ultrasonic blade 14 has at least one vibration node 34, and the sheath 32 is attached to the ultrasonic blade 14 proximate the at-least-one vibration node 34 of the attached ultrasonic blade 14. In one variation, the sheath 32 is flexible and does not extend to the housing 24. In one variation, the sheath 32 and the housing 24 are adapted to be held by a user when the distal blade portion 28 contacts and medically treats patient tissue. In one technique, the tube 16 is a relatively short tube, and the user manipulates the hand-held sheath 32 relative to the hand-held housing 24 to bend the tube 16 during a medical procedure to allow the ultrasonic blade 14 to more easily access a target site in a patient.

In a second employment, as shown in the alternate embodiment of FIG. 2, the sheath 132 of the ultrasonic medical instrument 110 surrounds and is attached to the tube 116. In one variation, the sheath 132 extends at least from the proximal blade end 126 to the housing 124. In one modification, the sheath 132 has a flexible portion 140 (which can extend the entire length of the sheath) and the sheath 132, but not the housing 124, is adapted to be held by a user when the distal blade portion 128 contacts and medically treats patient tissue. In one technique, the tube 116 is a relatively long tube, the housing 124 is located on the floor (or other suitable location) and the user manipulates the hand-held sheath 132 relative to the non-hand-held housing 124 to bend the tube 116 during a medical procedure to allow the ultrasonic blade 114 to more easily access a target site in a patient.

In a third employment, not shown, wherein the tube is a flexible tube and the sheath surrounds the tube and extends from at least the proximal blade end to the housing, the sheath is a remotely-controlled articulating sheath (similar to the insertion tube of a flexible endoscope) which is attached to the tube.

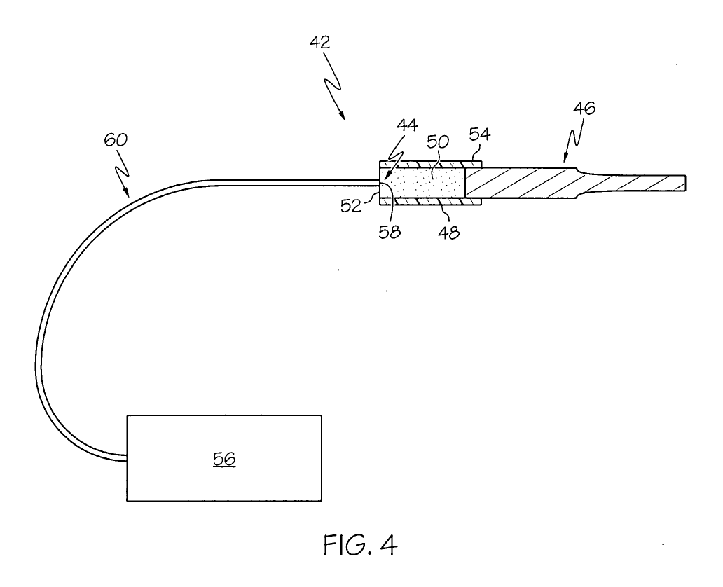

Referring again to the Figures, FIG. 4 illustrates a second embodiment of the invention. A first expression of the embodiment of FIG. 4 is for an ultrasound medical instrument 42 including a heat pulse initiator 44, a medical ultrasonic blade 46, and a tube 48. The heat pulse initiator 44 is adapted to output heat pulses having an ultrasonic pulse frequency. The tube 48 is adapted to contain a liquid 50 and has a first tube end 52 and a second tube end 54. The first tube end 52 is adapted to receive the heat pulses outputted by the heat pulse initiator 44. The ultrasonic blade 46 is acoustically connected to the liquid 50 proximate the second tube end 54, when the tube 48 contains the liquid 50. It is noted that the heat pulses have an energy sufficient to take the liquid 50 to its critical temperature, in turn causing the liquid 50 to expand and from a pressure wave which then creates an ultrasonically-vibrating energy wave in the liquid 50, as can be appreciated by those skilled in the art. In one implementation, the tube 48 is filled with the liquid 18. In one example, not shown, the tube is a flexible tube.

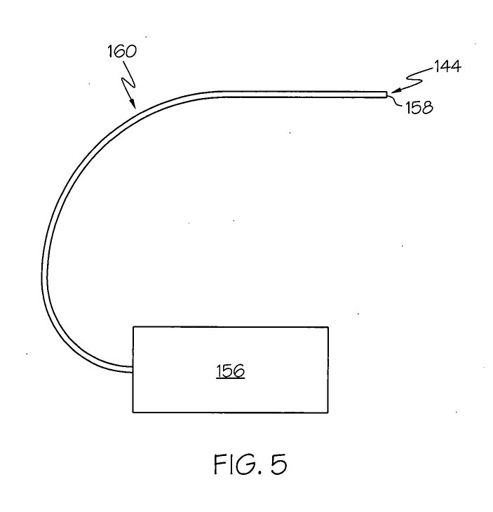

In one enablement of the first expression of the embodiment of FIG. 4, the tube 48 is a rigid tube. In application of the first expression of the embodiment of FIG. 4, the heat pulse initiator 44 is driven by an electric power source 56, and the heat pulse initiator 44 includes an end 58 of a resistive or a (bipolar) radio-frequency flexible wire 60 operatively connected to the electric power source 56. In one arrangement, not shown, the heat pulse initiator includes the ends of a plurality of resistive or radio-frequency flexible wires. In a different application, as shown in the alternate embodiment of FIG. 5, the heat pulse initiator 144 is driven by a laser light source 156, and the heat pulse initiator 144 includes an end 158 of a flexible laser fiber 160 operatively connected to the laser light source 156. In one arrangement, not shown, the heat pulse initiator includes the ends of a plurality of flexible laser fibers. Other heat pulse initiators are left to those skilled in the art.

It is noted that the variations, constructions, employments (including sheaths), etc. of the first embodiment of FIG. 1 and alternate embodiments of FIGS. 2 and 3 are equally applicable to the first expression of the second embodiment of FIG. 4 and the alternate embodiment of FIG. 5 substituting “heat pulse initiator” for “ultrasound transducer”.

Several benefits and advantages are obtained from one or more of the embodiments of the invention. In one example of the first and/or the second embodiment, the tube is a flexible tube and is controllably bent by a user during a medical procedure to allow the medical ultrasonic blade to more easily access a target site in a patient.

While the present invention has been illustrated by a description of several embodiments, it is not the intention of the applicant to restrict or limit the spirit and scope of the appended claims to such detail. Numerous other variations, changes, and substitutions will occur to those skilled in the art without departing from the scope of the invention. For instance, the ultrasonic medical instrument has application in robotic assisted surgery taking into account the obvious modifications of such systems, components and methods to be compatible with such a robotic system. It will be understood that the foregoing description is provided by way of example, and that other modifications may occur to those skilled in the art without departing from the scope and spirit of the appended Claims.

What is claimed is:

1. An ultrasonic medical instrument comprising:
a) an ultrasound transducer;
b) a medical ultrasonic blade; and
c) a tube adapted to contain a liquid and having a first tube end and a second tube end, wherein the first tube end is adapted to have the liquid proximate the first tube end, when the tube contains the liquid, be ultrasonically vibrated to generate an ultrasonically-vibrating energy wave, and wherein the ultrasonic blade is acoustically connected to the liquid proximate the second tube end, when the tube contains the liquid.

2. The ultrasonic medical instrument of claim 1, wherein the tube is a flexible tube and is filled with the liquid.

3. The ultrasonic medical instrument of claim 2, wherein the liquid is an at-least-partially de-gassed liquid.

4. The ultrasonic medical instrument of claim 2, wherein the liquid is a pressurized liquid.

5. The ultrasonic medical instrument of claim 2, wherein the liquid consists essentially of water or mineral oil.

6. The ultrasonic medical instrument of claim 2, wherein the ultrasonic blade includes a proximal blade end and a distal blade portion, and wherein the distal blade portion is adapted to contact and medically treat patient tissue.

7. The ultrasonic medical instrument of claim 6, wherein the ultrasonically-vibrating energy wave has a vibration antinode, and wherein the proximal blade end is disposed proximate the vibration antinode.

8. The ultrasonic medical instrument of claim 7, also including a housing containing the ultrasound transducer, wherein the tube is attached to the housing.

9. The ultrasonic medical instrument of claim 8, wherein the housing and the tube are adapted to be held by a user when the distal blade portion contacts and medically treats patient tissue.

10. The ultrasonic medical instrument of claim 9, also including an ultrasound generator disposed outside the housing and operatively connected to the ultrasound transducer.

11. The ultrasonic medical instrument of claim 8, wherein the tube, but not the housing, is adapted to be held by a user when the distal blade portion contacts and medically treats patient tissue.

12. The ultrasonic medical instrument of claim 11, also including an ultrasound generator disposed inside the housing and operatively connected to the ultrasound transducer.

13. The ultrasonic medical instrument of claim 2, wherein the tube has a shoulder between the first and second tube ends, wherein the tube has a first inside diameter between the first tube end and the shoulder, wherein the tube has a second inside diameter between the shoulder and the second tube end, and wherein the first diameter is not equal to the second diameter.

14. The ultrasonic medical instrument of claim 2, also including a sheath surrounding and attached to the ultrasonic blade.

15. The ultrasonic medical instrument of claim 2, also including a sheath surrounding and attached to the tube.

16. An ultrasonic medical instrument comprising:
a) a heat pulse initiator adapted to output heat pulses having an ultrasonic pulse frequency;
b) a medical ultrasonic blade; and
c) a tube adapted to contain a liquid and having a first tube end and a second tube end, wherein the first tube end is adapted to receive the heat pulses outputted by the heat pulse initiator, and wherein the ultrasonic blade is acoustically connected to the liquid proximate the second tube end, when the tube contains the liquid.

17. The ultrasonic medical instrument of claim 16, wherein the tube is filled with the liquid.

18. The ultrasonic medical instrument of claim 17, wherein the tube is a rigid tube.

19. The ultrasonic medical instrument of claim 17, wherein the heat pulse initiator is driven by an electric power source, and wherein the heat pulse initiator includes an end of a resistive or radio-frequency flexible wire operatively connected to the electric power source.

20. The ultrasonic medical instrument of claim 17, wherein the heat pulse initiator is driven by a laser light source, and wherein the heat pulse initiator includes an end of a flexible laser fiber operatively connected to the laser light source.
